(19) United States
(12) Patent Application Publication
(10) Pub. No.: US 2016/0302877 A1
(43) Pub. Date: Oct. 20, 2016

(54) OPHTHALMIC PROCEDURE ASSEMBLIES AND METHODS OF USING THE SAME

(71) Applicant: RELIANCE MEDICAL PRODUCTS, INC., Mason, OH (US)

(72) Inventors: Gary G. Schwaegerle, Cincinnati, OH (US); Chaz Strubbe, Loveland, OH (US); Abdoulaye Ba, Cincinnati, OH (US); Christopher Riemann, Cincinnati, OH (US)

(21) Appl. No.: 15/131,775

(22) Filed: Apr. 18, 2016

Related U.S. Application Data
(60) Provisional application No. 62/148,274, filed on Apr. 16, 2015.
(60) Provisional application No. 62/254,885, filed on Nov. 13, 2015.

(51) Int. Cl. A61B 50/33; A61B 50/36

(52) U.S. Cl. CPC A61B 50/33; A61B 50/362; A61B 2050/3008

(57) ABSTRACT

A tray cover for securing one or more accessories to an instrument tray, the tray cover including a plurality of wells, wherein, when the tray cover is placed on the instrument tray, each of the one or more accessories is engaged by one of the plurality of wells and is secured to the instrument tray by the tray cover.

### CROSS-REFERENCE TO RELATED APPLICATIONS

This application is related to and claims priority to U.S. Provisional Patent Application Ser. No. 62/148,274, which was filed on Apr. 16, 2015 and U.S. Provisional Patent Application Ser. No. 62/254,885, which was filed on Nov. 13, 2015. The complete and entire disclosures of each of these applications are hereby expressly incorporated by reference herein.

### TECHNICAL FIELD

The present teachings are generally related to instrument tray assemblies and accessories for use during a medical procedure, and more particularly to instrument tray assemblies and accessories used in ophthalmic procedures.

### BACKGROUND OF THE DISCLOSURE

The statements in this section merely provide background information related to the present disclosure and should not be construed as constituting prior art.

During an ophthalmic procedure, a wide variety of instruments and supplies may be used by the doctor or technician. Commonly used materials include anesthetics, antibiotics, and various medications. The methods of applying such materials are diverse. For example, gel packs, dropper bottles, and syringes may be used to apply or deliver compositions during a procedure. Other supplies such as sterile cotton swabs and tissues are frequently used as well. Typically, a technician will set up the required supplies on a desk or Mayo tray stand in the treatment room. There may be significant variation in the arrangement of the materials based on the preferences of the technician and/or the doctor. During the procedure, the doctor may stand between the patient and the desk in order to have access to the instruments and materials. The technician may typically retrieve the supplies and hand them to the doctor. If a technician is not assisting the doctor, the doctor frequently has to turn away from the patient to retrieve the next needed supply. Two hands are often required to handle the supplies. For instance, using a syringe to inject a composition into the eye requires both hands to remove the cap.

There are significant efficiency drawbacks to current methods of arranging and utilizing supplies during ophthalmic procedures. For instance, because the order of the supplies may change depending on who sets them up, time is wasted as the doctor or technician finds a needed supply or instrument. Further, there is wasted movement as, for instance, the technician uses both hands to remove a cap from a syringe and then hands the syringe to the doctor. Such double handling of items increases the time for each procedure, thus reducing the efficiency. Even if the doctor removes the cap from the syringe, the doctor still must use both hands to do so. In this regard, the doctor is prevented from maintaining contact with the patient, which may increase the patient's anxiety during the procedure. Also, increased handling of an uncapped syringe and disposing of the syringe increases the likelihood of unintentional contact with the needle.

There is a need, therefore, for an assembly that addresses one or more of the needs in the field of ophthalmic examination and treatment and provides for improved positioning of materials used during a procedure.

### SUMMARY OF THE DISCLOSURE

In one embodiment, a tray cover for securing one or more accessories to an instrument tray includes a plurality of wells. When the tray cover is placed on the instrument tray, each of the one or more accessories is engaged by one of the plurality of wells and is secured to the instrument tray by the tray cover.

In one embodiment, an instrument tray assembly for use during a medical procedure includes an instrument tray and a tray cover having a plurality of wells. The tray cover is configured to be placed on the instrument tray. When one or more accessories are positioned on the instrument tray and the tray cover is placed on the instrument tray, each of the one or more accessories is engaged by one of the plurality of wells and is secured to the instrument tray by the tray cover.

In one embodiment, an instrument tray assembly for use during a medical procedure includes a base tray, an instrument tray, a tray cover having a plurality of wells, and a locking mechanism configured to secure the tray cover to the base tray. The instrument tray is configured to be positioned on the base tray. The tray cover is configured to be positioned on the instrument tray. When one or more accessories are positioned on the instrument tray and the tray cover is positioned on the instrument tray, each of the one or more accessories is engaged by one of the plurality of wells and is secured to the instrument tray by the tray cover.

In one embodiment, an instrument stand assembly for use during a medical procedure includes a base, a support arm extending from the base, a base tray removably mounted to the support arm, an instrument tray configured to be placed on the base tray, a tray cover having a plurality of wells, and a locking mechanism configured to secure the tray cover and the instrument tray to the base tray. The tray cover is configured to be placed on the instrument tray. When one or more accessories and the tray cover are positioned on the instrument tray and the tray cover is secured to the base tray, each of the one or more accessories is engaged by one of the plurality of wells and is secured to the instrument tray by the tray cover.

In one embodiment, an instrument stand assembly for use during a medical procedure includes a base and a support arm extending from the base. The support arm has a first end and a second end. The first end is coupled to the base, and the support arm is configured to allow the second end to articulate relative to the first end. The support arm is configured to support one or more accessories and retain an orientation of the one or more accessories relative to the base when the second end articulates.

In one embodiment, an accessory holder includes a body configured to support an accessory. When the accessory holder is coupled to a support arm extending from a base and the support arm articulates, an orientation of the accessory holder relative to the base is retained. In other words, the orientation of the accessory holder remains constant during articulation or pivoting of the support arm up and down along a pivot axis.

In one embodiment, a syringe holder includes a housing having one or more cavities, each of the one or more cavities is configured to receive a syringe cap, an engagement member having an unengaged position and an engaged position, and an actuator configured to move the engagement member from the unengaged position to the engaged position. When one of the cavities receives the syringe cap and the engagement member is in the engaged position, the engagement member is configured to secure the syringe cap in the cavity.

In one embodiment, a container holder includes a plurality of engagement members configured to engage a cap. When the cap is engaged and a container coupled to the cap is being rotated, the plurality of engagement members substantially prevent the cap from rotating.

In accordance with yet another embodiment, a method of securing one or more accessories to an instrument tray using a tray cover is provided. In accordance with this method, the tray cover has a plurality of wells and comprises the steps of: positioning one or more accessories on the instrument tray; positioning the tray cover on the instrument tray over the one or more accessories, each of the one or more accessories being engaged by one of the plurality of wells; and securing the tray cover to the instrument tray using a locking mechanism to prevent relative movement between the one or more accessories and the instrument tray.

In accordance with still another embodiment, a method of securing one or more accessories to an instrument tray using a tray cover is provided. In accordance with this method, the tray cover has a plurality of wells and comprises the steps of: positioning one or more accessories on the instrument tray; positioning the tray cover on the instrument tray over the one or more accessories, each of the one or more accessories being engaged by one of the plurality of wells; positioning the instrument tray on a base tray; and securing the tray cover to the base tray using a locking mechanism to prevent relative movement between the one or more accessories and the base tray.

According to yet another embodiment herein, a method of uncoupling a syringe from a syringe cap using a syringe holder is provided. In accordance with this method, the syringe holder comprises a housing having one or more cavities and an actuator and includes the steps of: positioning the syringe cap in one of the one or more cavities; and engaging the actuator to secure the syringe cap in the cavity. When the syringe is uncoupled from the syringe cap, the syringe cap remains secured in the cavity.

In accordance with still one additional embodiment of the present disclosure, a method of uncoupling a container from a container cap using a container holder is provided. According to this embodiment, the container holder comprises a plurality of engagement members, and the method includes the steps of: positioning the container cap in the container holder; and securing the container cap to the container holder using the plurality of engagement members. When the container is uncoupled from the container cap, the container cap remains secured to the container holder.

Other objects and benefits of the disclosure will become apparent from the following written description along with the accompanying drawings.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure taken in conjunction with the accompanying drawings, wherein:

FIG. 1 is a perspective view of an assembly according to one embodiment of the present disclosure.

FIG. 2 is a perspective view of a portion of the assembly of FIG. 1.

FIGS. 3A and 3B are side views of a portion of the assembly of FIG. 1 and respectively showing an instrument arm in different positions.

FIG. 3C is a cross-sectional view of a portion of the assembly taken along section line 3C-3C of FIG. 3B.

FIG. 4 is a perspective view of a portion of an assembly according to another embodiment of the present disclosure.

FIG. 5 is a perspective view of a portion of the assembly of FIG. 1.

FIG. 6 is an exploded view of the assembly of FIG. 1.

FIGS. 7A and 7B are cross-sectional views of a portion of the assembly taken along section line 7-7 of FIG. 5 and respectively showing brackets in different positions.

FIG. 8 is a perspective view of a tray cover according to one embodiment of the present disclosure.

FIG. 9 is a perspective view of a syringe holder according to one embodiment of the present disclosure.

FIGS. 10A and 10B are cross-sectional views of a portion of the syringe holder taken along section line 10-10 of FIG. 9 and respectively showing an actuator in different positions.

FIG. 11 is a perspective view of a container holder according to one embodiment of the present disclosure.

FIGS. 12A and 12B are top views of a portion of the container holder taken along section line 12-12 of FIG. 11 and respectively showing spindles in different positions.

FIG. 13 is a perspective view of a container holder according to another embodiment of the present disclosure.

FIGS. 14A and 14B are top views of a portion of the container holder taken along section line 14-14 of FIG. 13 and respectively showing clamps and a lever in different positions.

FIG. 15 is a perspective view of a container holder according to another embodiment of the present disclosure.

FIG. 16 is an exploded perspective view of a container holder according to another embodiment of the present disclosure.

FIG. 17 is an assembled perspective view showing the container holder of FIG. 16, with the housing or knob in a raised position.

FIG. 18 is a cross sectional view taken along line 18-18 of FIG. 17, but eliminating internal components for clarity.

FIG. 19 is a perspective view similar to FIG. 17, but illustrating the housing or knob in a depressed, unlocked position.

FIG. 20 is a cross sectional view taken along line 20-20 of FIG. 19, and again with internal components eliminated for clarity.

FIG. 21 is a perspective view similar to FIG. 19, but schematically illustrating rotation of the housing or knob to a clamped position.

FIGS. 22A and 22B are cross sectional views taken along line 22B-22B of FIG. 21 but respectively illustrating unclamped and clamped positions of the clamps during rotation of the housing or knob.

FIG. 23 is a partial perspective view illustrating another alternative embodiment of a well cover in accordance with the present disclosure.

FIG. 24 is a partial perspective view illustrating another alternative embodiment of a bracket in accordance with the present disclosure.

### DETAILED DESCRIPTION

The above-mentioned aspects of the present application and the manner of obtaining them will become more apparent and the teachings of the present application itself will be better understood by reference to the following description of the embodiments of the present application taken in conjunction with the accompanying drawings.

The embodiments of the present application described below are not intended to be exhaustive or to limit the teachings of the present application to the precise forms disclosed in the following detailed description. Rather, the embodiments are chosen and described so that others skilled in the art may appreciate and understand the principles and practices of the present application.

Unless defined otherwise, all technical and scientific terms used herein have the same meaning as commonly understood by one of ordinary skill in the art to which this application belongs. Although any method and materials similar or equivalent to those described herein can be used in the practice or testing of the present application, the specific methods and materials are now described.

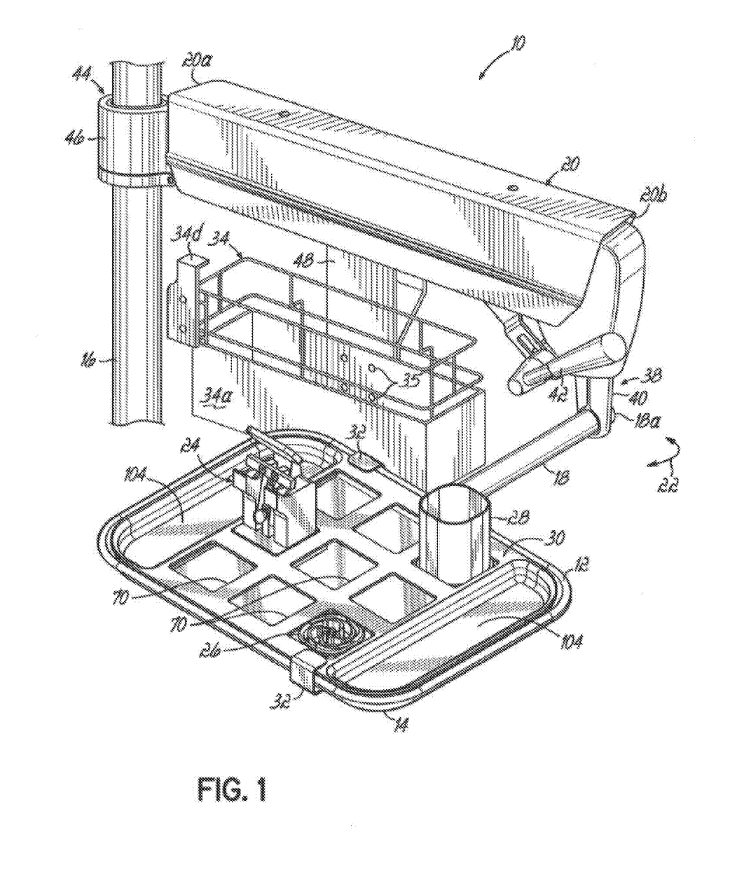

Referring first to FIG. 1, an ophthalmic instrument stand assembly 10 constructed in accordance with an illustrative embodiment of the invention is shown. Assembly 10 includes an instrument tray 12, which is supported by a base tray 14. Base tray 14 is movably coupled to a support pole 16. Base tray 14 is coupled to support pole 16 by instrument arms 18, 20. Base tray 14 is rotatably mounted on instrument arm 18. Instrument arm 18 is pivotally coupled to instrument arm 20. Instrument arm 20 is slidably mounted on support pole 16. Instrument arms 18, 20 are adjustable, such that instrument tray 12 is capable of being rotated toward and away from the operative position indicated by arrow 22. Further, the height of instrument tray 12 is adjustable. For instance, instrument arm 20 is configured to vertically pivot relative to pole 16. A variety of accessories, which are described below, are positioned on the tray. For example, a syringe holder 24, container holder 26, and cup 28 are shown on the instrument tray 12. The container, for example, may be a bottle, a gel pack, or any other container for holding a substance used during a procedure.

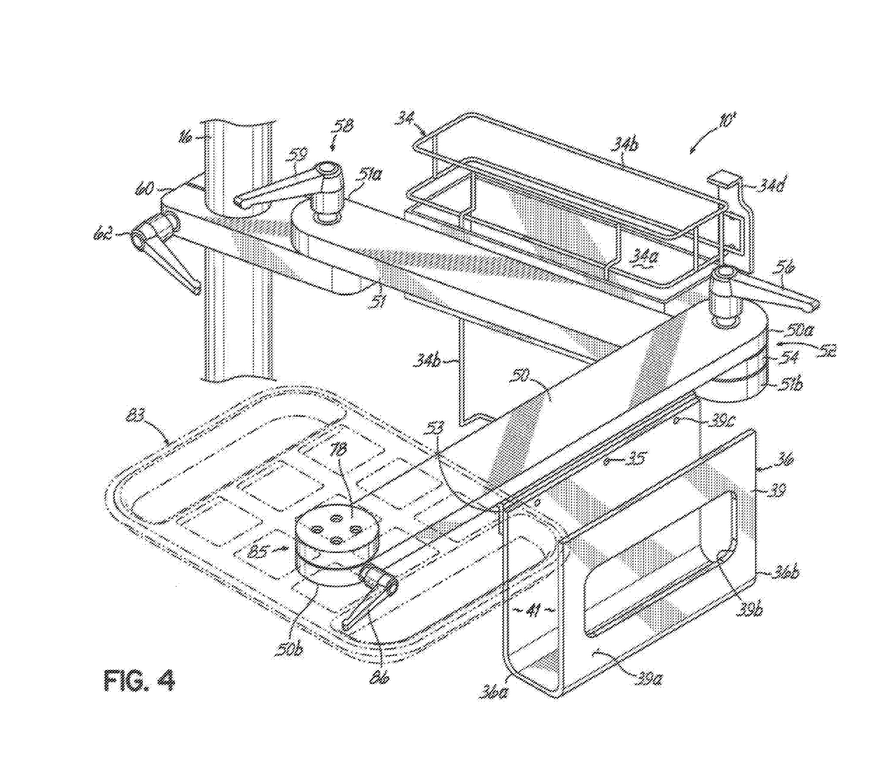

A tray cover 30 is positioned over the syringe holder 24, container holder 26, and cup 28. Tray cover 30 is secured to base tray 14 by brackets 32, which are slidably connected to base tray 14. As will be described in detail, tray cover 30 and brackets 32 together secure the accessories to instrument tray 12 and base tray 14. Assembly 10 may also include a variety of accessories connected to arms 18, 20. For example, these accessories may include a sharps disposal container holder 34 and a tissue box holder 36 (shown in FIG. 4).

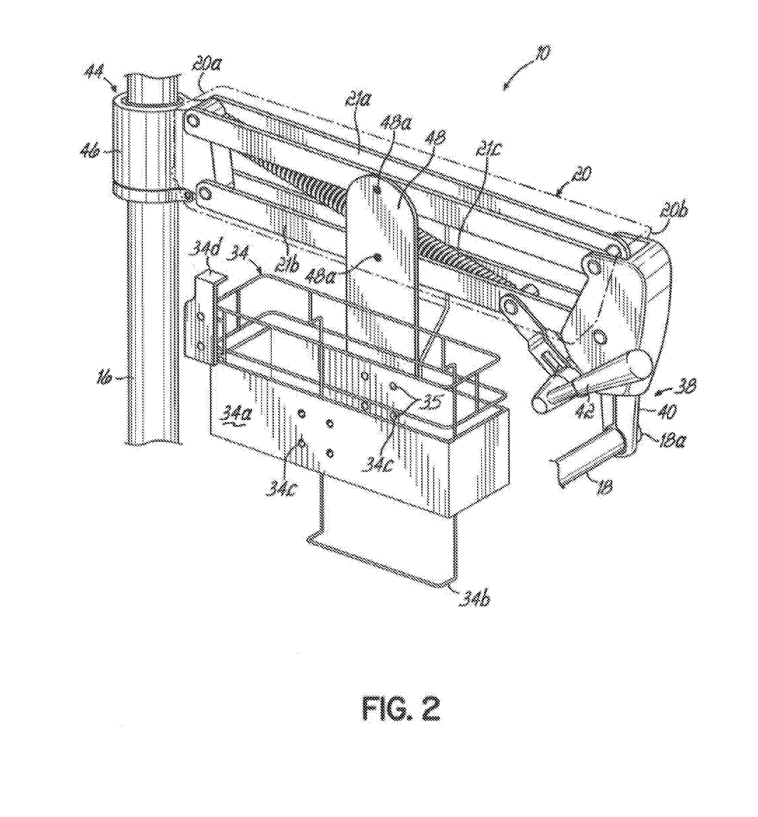

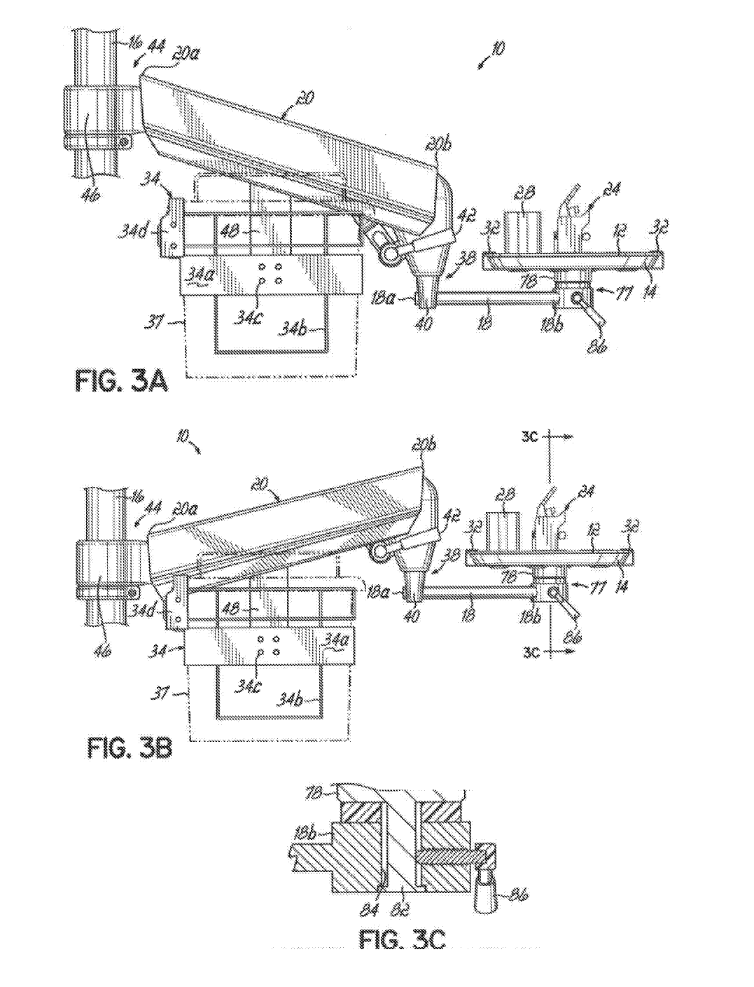

Now referring to FIGS. 2, 3A, and 3B, arms 18, 20 are shown in more detail. Arms 18, 20 are pivotally coupled at pivot connection 38. Pivot connection 38 is made by way of end 18a of arm 18 extending through connection member 40, which is coupled to end 20b of arm 20. Lever 42 is configured to lock the rotation of arm 18 relative to arm 20. Arm 20 is pivotally coupled to pole 16 at pivot connection 44. Pivot connection 44 is made by way of pole 16 extending though connection member 46, which is coupled to end 20a of arm 20. Although not shown, there may be a mechanism configured to lock the rotation of arm 20 relative to pole 16. Arm 20 includes an upper link member 21a, a lower link member 21b, and a counterbalancing spring 21c. In this configuration, arm 20 is capable of articulating relative to pole 16, such that end 20b of arm 20 is raised and lowered relative to end 20a. Further, pivot connection 38 may remain in the same configuration when end 20b of arm 20 is raised or lowered. In other words, pivot connection 38 is configured to prevent any rotational movement of arm 18 when arm 20 is articulated. Lever 42 is also configured to lock the articulation of arm 20. An arm capable of such articulation is described in U.S. Pat. No. 5,907,387, which is incorporated by reference herein in its entirety. Additionally, sharps disposal container holder 34 is coupled to arm 20 by a member 48. Member 48 is coupled to the upper and lower links 21a, 21b via pivot links 48a to create a four bar linkage. When end 20b of arm 20 is raised or lowered, the connection between arm 20 and member 48 is configured to prevent any rotational movement of sharps disposal container holder 34, as shown in a comparison of FIGS. 3A and 3B. In other words, the orientation of sharps disposal container holder 34 relative to, for example, the support pole 16 does not change when arm 20 is articulated up and down about a pivot joint generally located at end 20a. Instead, the container holder 34 remains in a horizontal orientation, as shown, during the articulation of arm 20 from its lower position (FIG. 3A) to its upper position (FIG. 3B).

With reference to FIG. 4, an ophthalmic instrument assembly 10′ constructed in accordance with an illustrative embodiment of the invention is shown. Arm 50 is pivotally coupled to arm 51 at pivot connection 52. Pivot connection 52 is made by way of end 50a of arm 50 being coupled to end 51b of arm 51 by connection member 54. Lever 56 is configured to lock the rotation of arm 50 relative to arm 51. Arm 51 is pivotally coupled to pole 16 at pivot connection 58. Pivot connection 58 is made by way of pole 16 extending though connection member 60, which is pivotally coupled to end 51a of arm 51. Lever 59 is configured to lock the rotation of arm 51 relative to connection member 60. Pole 16 is slidable through connection member 60. Lever 62 is configured to lock the movement of connection member 60 relative to pole 16. Additionally, tissue box holder 36 is coupled to arm 50 by a connection member 53.

Referring again to FIGS. 2-4, exemplary accessories attached to arms 18, 20 and arms 50, 51 are shown in detail. Sharps disposal container holder 34 includes a rim 34a and a wire cage 34b. Rim 34a includes bores 34c and is coupled to connection member 48 via fasteners 35 (FIG. 2). A disposal container, such as a sharps container 37 (shown in phantom in FIGS. 3A and 3B), may be placed in sharps disposal container holder 34. Sharps disposal container holder 34 is configured to lock sharps container 37 in place via locking mechanism 34d. Rim 34a may be used to support another accessory, such as a tissue box holder (not shown), via bores 34c. With specific reference to FIG. 4, tissue box holder 36 includes a body 39 with a U-shaped cross-section that defines a cavity 41 open to first and second ends 36a, 36b of tissue box holder 36. A front surface 39a of tissue holder 36 includes an aperture 39b. A tissue box (not shown) may be placed in cavity 41, and a tissue may be pulled from tissue box through aperture 39b. Tissue box holder 36 further includes bores 39c and is coupled to connection member 53 via fasteners 35. Using an instrument arm to support accessory holders such as sharps disposal container holder 34 and tissue box holder 36 may reduce inefficiencies and increase the safety of a procedure. For instance, having a sharps container 37 in close proximity to the patient allows for quick disposal of a used syringe.

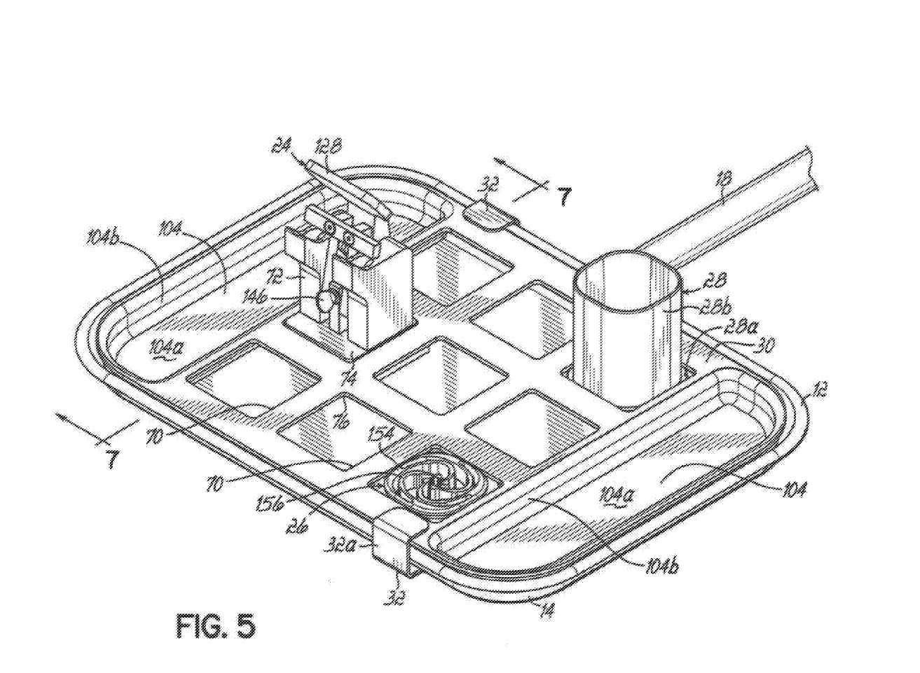

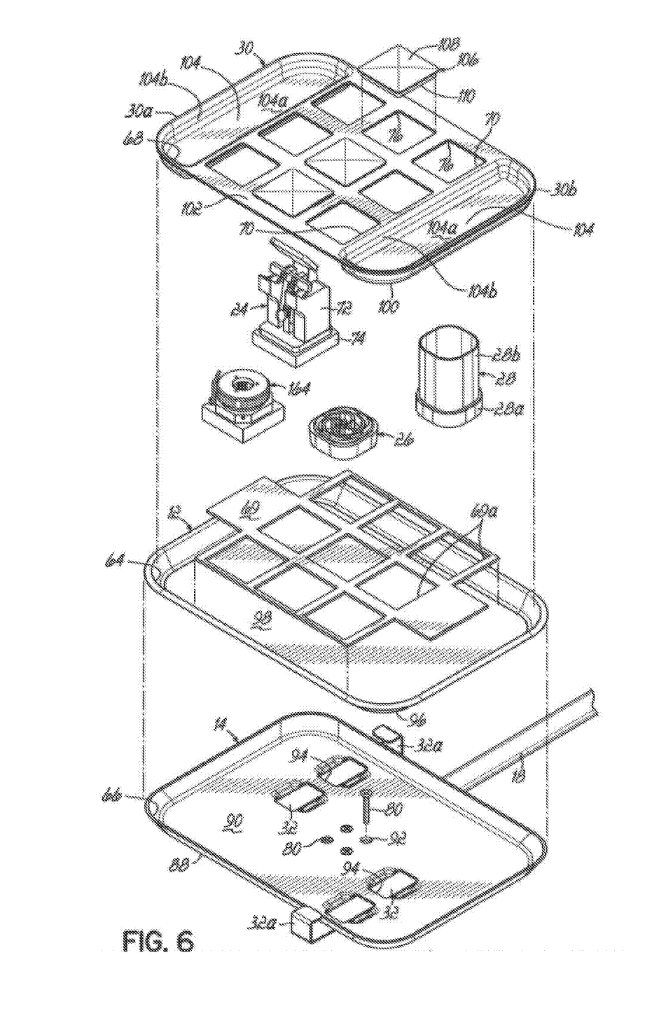

Referring now to FIGS. 5 and 6, the base tray 14, instrument tray 12, and tray cover 30 are illustrated in more detail. A peripheral side wall 64 of instrument tray 12 is positionable within a peripheral side wall 66 of base tray 14. Similarly, a peripheral side wall 68 of tray cover 30 is positionable within the instrument tray side wall 64. Tray cover 30 includes a plurality of wells 70. A locator 69 is included and sits within the instrument tray 12, between the instrument tray 12 and tray cover 30. This locator 69 includes cutouts 69a that corresponds to various accessories and that will locate the various accessories such that they accurately extend into the wells 70 when the tray cover 30 is placed within the instrument tray 12. The accessories on instrument tray 12 extend through wells 70 and are engaged with the wells 70. For example, a housing 72 of syringe holder 24 may pass through one of the wells 70, while a base 74 of syringe holder 24 is engaged with a wall 76 of one of the wells 70. Brackets 32 are configured to frictionally engage tray cover 30 when tray cover 30 and instrument tray 12 are positioned on base tray 14.

With reference to FIGS. 3A-3C and 6, the connection between base tray 14 and arm 18 is shown in more detail. Base tray 14 is connected to arm 18 at pivot connection 77. Pivot connection 77 is made by way of base tray 14 being coupled to a swivel base 78 by fasteners, such as screws 80. Swivel base 78 is rotatably coupled to end 18b of arm 18. In this regard, swivel base 78 includes a pin 82 secured thereto in a rigid manner such that pin 82 extends vertically downward. Pin 82 is received within a bore 84 in an end 18b of arm 18 (FIG. 3C). Thus, as base tray 14 and swivel base 78 are rotated relative to arm 18, pin 82 rotates within bore 84. Lever 86 is configured to lock the rotation of swivel base 78 relative to arm 18.

With reference again to FIG. 4, another configuration is shown similar to the connection shown in FIGS. 3A-3C and 6. A tray assembly 83 is connected to arm 50 at pivot connection 85. Pivot connection 85 is made by way of tray assembly 83 being coupled to swivel base 78 and swivel base 78 being rotatably coupled to end 50b of arm 50. Lever 87 is configured to lock the rotation of swivel base 78 relative to arm 50.

Referring again to FIG. 6, base tray 14 is shown in more detail. Base tray 14 includes a bottom surface 88, top surface 90, and peripheral side wall 66. Base tray 14 further includes a plurality of bores 92 through which screws 80 fasten base tray 14 to swivel base 78. Base tray 14 includes a plurality of recessed slots 94. Slots 94 slidably receive brackets 32. The connection between base tray 14 and brackets 32 is further described below.

Referring still to FIG. 6, instrument tray 12 is shown in more detail. Instrument tray 12 includes a bottom surface 96, top surface 98, and peripheral side wall 64. When instrument tray 12 is positioned in base tray 14, side wall 64 may align with side wall 66 of base tray 14. Further, bottom surface 96 may contact and be supported by top surface 90 of base tray 14. Additionally, side wall 66 of base tray 14 is configured to be flush with the outer edge of side wall 64 of instrument tray 12. Alternatively, side wall 66 of base tray 14 may be configured to cover an edge of instrument tray side wall 64 when instrument tray 12 is positioned in base tray 14. In this regard, if the edge of instrument tray 12 is sharp, base tray 14 may provide protection against unintentional contact with the sharp edge. In one embodiment, instrument tray 12 is a Mayo tray, which is a stainless steel instrument tray.

With further reference to FIG. 6, tray cover 30 is shown in more detail. Tray cover 30 includes a bottom surface 100, top surface 102, and peripheral side wall 68. Tray cover 30 further includes the plurality of wells 70. Each well 70 has a square configuration although any suitable shape or configuration may be used in addition to or instead of those shown. In one embodiment, a well 70 is about 2 inches wide and about 2 inches long. The plurality of wells 70 are arranged in a square matrix having three columns and three rows. When tray cover 30 is placed over accessories that are positioned on instrument tray 12, wells 70 are configured to engage the accessories. Tray cover 30 further includes a plurality of recesses 104. Recesses 104 extend along opposing ends 30a, 30b of tray cover 30. The doctor or technician may utilize recesses 104 to hold miscellaneous supplies. For instance, a packet of cotton swabs may be placed in a recess 104. Each recess 104 includes a bottom surface 104a and a wall 104b. In this configuration, side wall 68 of tray cover 30 includes portions of walls 104b. When tray cover 30 is positioned on instrument tray 12, portions of walls 104b may be in contact with side wall 64 of instrument tray 12. Moreover, wells 70 are spaced apart from the top surface 98 of instrument tray 12.

Instrument tray 12 and/or tray cover 30 may be configured to be cleaned in an autoclave. In this regard, instrument tray 12 and/or tray cover 30 may be formed from materials that will not be deformed or damaged at conventional autoclave temperatures. Alternatively, instrument tray 12 and/or tray cover 30 may be configured to be cleaned using, for example, an alcohol bath or water and soap suitable for sanitizing medical instruments.

Still referring to FIG. 6, the cup 28 is shown. Cup 28 includes a base 28a and a cup wall 28b extending from base 28a. Base 28a has a cross-section that is larger than the size of wells 70 such that base 28a will not pass through the wells 70. Cup wall 28b has a cross-section that will pass through the wells 70. Cup 28 may be configured to be cleaned using, for example, an alcohol bath, water and soap suitable for sanitizing medical instruments, or an autoclave.

Referring still to FIG. 6, a plurality of well covers 106 are shown. Each well cover 106 is configured to cover a well 70 when the well 70 is not engaged with an accessory. Each well cover 106 may include a top surface 108 and a side wall 110. The top surface 108 has a cross-section that is larger than the size of wells 70 such that top surface 108 will not pass through the wells 70. The side wall 110 has a cross-section that is smaller than the size of wells 70 such that side wall 110 will pass through the wells 70. When a well cover 106 is positioned in a well 70, side wall 110 is frictionally engaged with wall 76 of one of the wells 70. In this regard, when tray cover 30 is secured to instrument tray 12 by brackets 32, well cover 106 is secured in position in well 70, which may advantageously prevent contaminants or loose supplies from falling within the well 70.

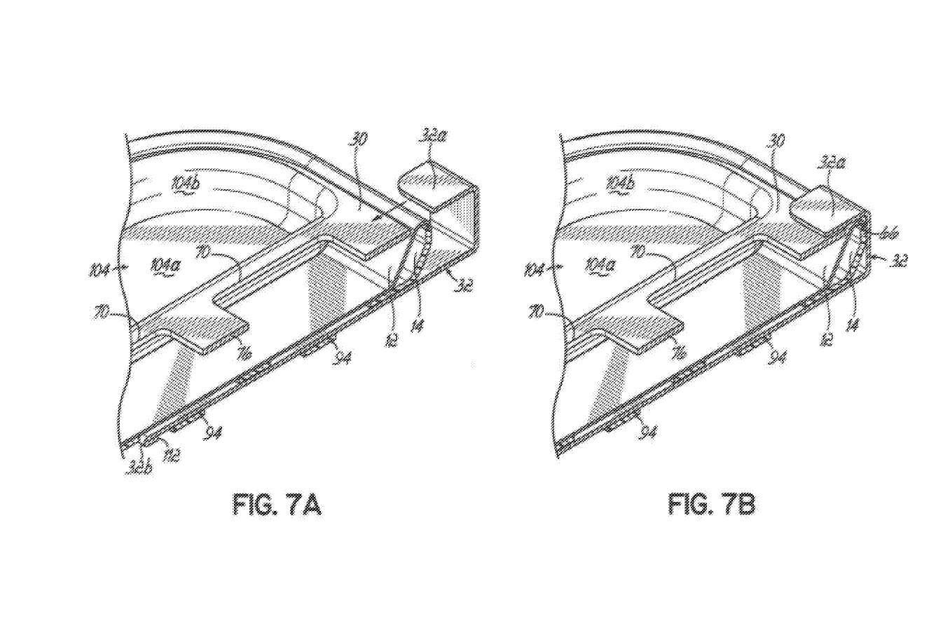

Now referring to FIGS. 7A and 7B, the connection between base tray 14 and brackets 32 is shown in more detail. Brackets 32 may be in a first, unengaged position where an end 32a is not in contact with instrument tray 12 or tray cover 30 (FIG. 7A). Brackets 32 may slide to a second, engaged position where end 32a is in contact with tray cover 30 (FIG. 7B). In the engaged position, end 32a of brackets 32 are in contact with instrument tray 12 and side wall 66 of base tray 14. End 32a prevents tray cover 30 from being separated from instrument tray 12. When tray cover 30 is positioned around accessories, locking tray cover 30 relative to instrument tray 12 prevents movement of the accessories. After the procedure is complete, for example, brackets 32 may be slid to the unengaged position where tray cover 30 may be removed from instrument tray 12. As best seen in FIG. 7A, a lip 112 extends from an end 32b of each bracket 32. Lip 112 prevents brackets 32 from sliding beyond a predetermined location, such as one of the slots 94. Lip 112 prevents accidental disconnection of brackets 32 from base tray 14.

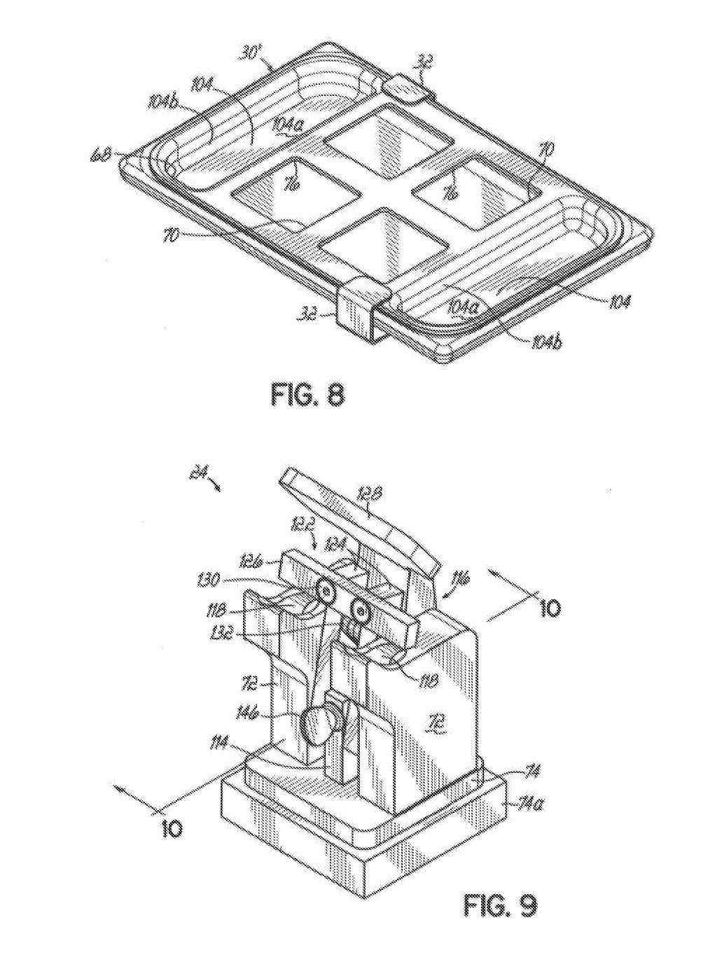

Referring now to FIG. 8, a tray cover 30′ is shown having a different configuration compared to tray cover 30 in FIGS. 5 and 6. Here, tray cover 30′ includes four wells 70 arranged in a square matrix having two columns and two rows.

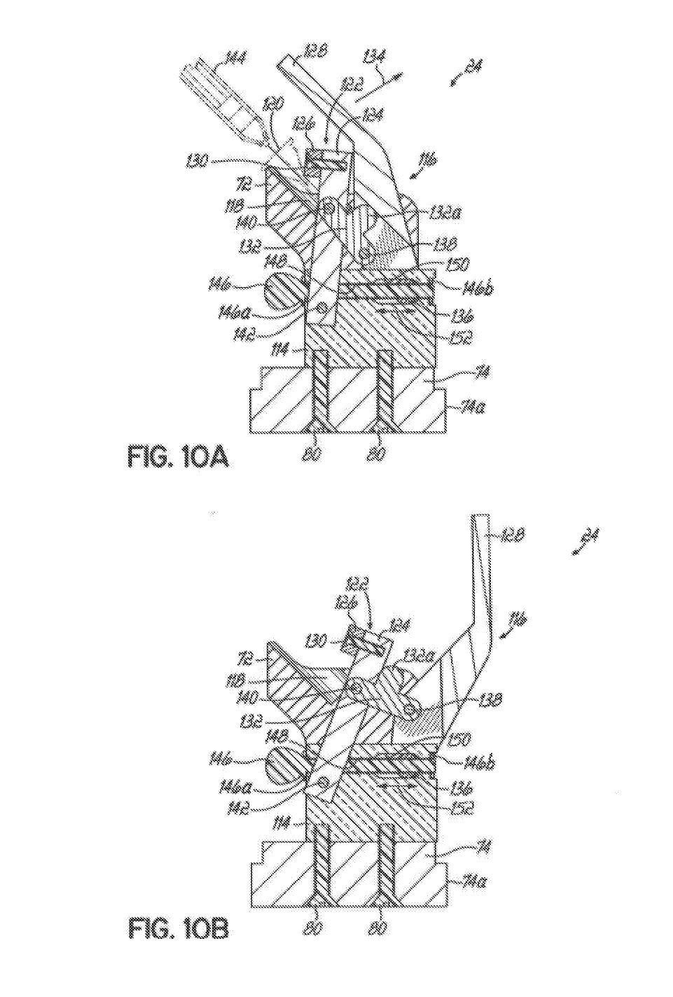

Turning now to FIGS. 9, 10A, and 10B, syringe holder 24 is shown in more detail. Syringe holder 24 includes a base 74, at least one housing 72, and an actuator 116. Actuator 116 includes an actuator base 114. Actuator base 114 is coupled to base 74 via fasteners such as a pair of screws 80. Each housing 72 is similarly coupled to base 74 via a pair of screws 80 (not shown). A section 74a of base 74 has a cross-section that is larger than the size of wells 70 such that base section 74a will not pass through the wells 70. In one embodiment where wells 70 have a width and a length of about 2 inches, base section 74a may have a width and a length of about 2.25 inches. Except for base section 74a, syringe holder 24 has a cross-section that is smaller than the size of wells 70 such that, for example, housings 72 and actuator 116 will pass through the wells 70. Housings 72 include one or more cavities 118. Each cavity 118 is configured to receive a syringe cap 120 (seen in phantom in FIGS. 10A and 10B).

With further reference to FIGS. 10A and 10B, actuator 116 includes a T-bar 122. T-bar 122 includes two arms 124 connected by a cross-bar 126 via fasteners such as screws 130. Actuator 116 further includes a handle 128 operably coupled to T-bar 122 via a pivot member 132. Handle 128 and T-bar 122 are pivotable relative to housing 72 such that T-bar 122 is capable of being rotated toward and away from the operative position indicated by arrow 134. More specifically, handle 128 is pivotally connected to actuator base 114 via pin 136. The position of pin 136 relative to actuator base 114 is adjustable, which is further discussed below. Handle 128 is pivotally coupled to pivot member 132 via fasteners such as pin 138. Note that FIGS. 10A and 10B show only one portion of actuator 116. Pivot member 132 is pivotally coupled to arms 124 of T-bar 122 via fasteners such as pin 140. Arms 124 of T-bar 122 are pivotally coupled to actuator base 114 via fasteners such as pin 142. In this manner, the connection between actuator base 114, handle 128, T-bar 122, and pivot member 132 acts as a four-bar linkage coupled by pins 136, 138, 140, 142. Handle 128 has a first, unengaged position where T-bar 122 is spaced apart from cavities 118 (FIG. 10B). Handle 128 may pivot to a second, engaged position (FIG. 10A). When handle 128 moves to its engaged position, pins 136, 138, 140, 142 move relative to each other and cross-bar 126 of T-bar 122 may move to at least partially cover each cavity 118. In the engaged position, pin 138 is positioned directly below the plane between pins 136, 140. Because pins 136, 138, 140, 142 move relative to each other, the position of pivot member 132 also changes relative to handle 128 and T-bar 122. In this regard, a rounded protrusion 132a moves into contact with handle 128. When handle 128 reaches the engaged position, a ‘click’ may be felt by the user as protrusion 132a comes into contact with handle 128.

Syringe holder 24 also includes an engagement member that, when syringe cap 120 is positioned in one of the cavities 118 and handle 128 is in the engaged position, is configured to secure syringe cap 120 relative to cavity 118 (FIG. 10B). In the illustrated embodiment, the cross-bar 126 of T-bar 122 acts as the engagement member. Thus, a syringe 144 may be uncoupled from syringe cap 120 while syringe cap 120 remains secured in cavity 118 by cross-bar 126. When syringe holder 24 is secured to instrument tray 12 by tray cover 30, the doctor may remove syringe 144 from cap 120 with one hand, which advantageously allows the doctor to use the other hand to, for example, hold open an eyelid of the patient. After the procedure is complete, handle 128 may be moved to its unengaged position where cross-bar 126 is no longer securing syringe cap 120 in cavity 118. Syringe cap 120 may then be removed from cavity 118.

Actuator 116 may also include an adjustment mechanism, such as adjustment screw 146, coupled to handle 128. Adjustment screw 146 is configured to adjust a position of the engagement member. Adjustment screw 146 is threaded and is received in a bore 148 in actuator 116. Bore 148 opens to a cavity 150. Pin 136 extends through cavity 150 and includes threads configured to mate with adjustment screw 146. As adjustment screw 146 rotates within bore 148, pin 136 moves within cavity 150, as can be seen by arrow 152. As pin 136 moves within cavity 150, handle 128 pivots. As handle 128 pivots, the T-bar 122 pivots causing the cross-bar 126 to move towards or away from cavities 118. In this manner, the location of the unengaged and engaged positions of the engagement member, cross-bar 126, may be adjusted by rotating adjustable screw 146. Adjusting the location of the unengaged and engaged positions advantageously allows cross-bar 126 to engage with syringe caps of differing sizes. A flange 146a and a retention ring 146b prevent unintentional movement of adjustment screw 146 through bore 148.

Syringe holder 24 may be configured to be cleaned in an autoclave. In this regard, syringe holder 24 may be formed from materials that will not be deformed or damaged at conventional autoclave temperatures. In one embodiment, syringe holder 24 is made of aluminum.

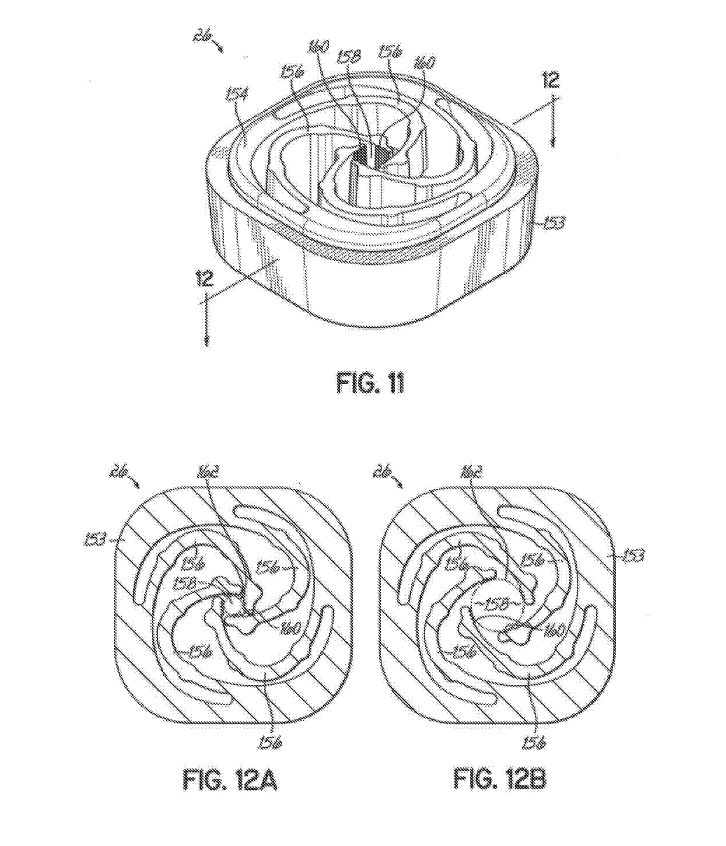

Referring now to FIGS. 11, 12A, and 12B, one embodiment of a container holder 26 is shown in more detail. Container holder 26 includes a base 153 and an outer rim 154 coupled to a plurality of engagement members, or spindles 156. Base 153 has a cross-section that is larger than the size of wells 70 such that base 153 will not pass through the wells 70. In one embodiment where wells 70 have a width and a length of about 2 inches, base 153 may have a width and a length of about 2.25 inches. The outer rim 154 defines a compartment 158. Spindles 156 are coupled to outer rim 154 and extend towards a center of compartment 158 in a clockwise spiral pattern. Each spindle 156 may include ridges 160. Container holder 26 is configured to hold a container cap 162 (shown in phantom in FIGS. 12A and 12B). Spindles 156 are configured to contact and frictionally engage container cap 162 when container cap 162 is positioned in compartment 158. Further, spindles 156 are flexible to allow container caps 162 of different sizes to be positioned in compartment 158, as shown by the different size container caps 162 shown in FIGS. 12A and 12B. When container cap 162 is engaged in container holder 26 and a container coupled to container cap 162 by mating threads is being rotated, spindles 156 substantially prevent container cap 162 from rotating (not shown). In this regard, the clockwise spiral configuration of the spindles 156 counteracts the counter-clockwise rotation of the container and causes the spindles 156 to grip the cap 162 tighter. When container holder 26 is secured to instrument tray 12 by tray cover 30, the doctor may remove the container from cap 162 with one hand, which advantageously allows the doctor to be free to use the other hand to, for example, hold open an eyelid of the patient. If the container is multi-use, the container may be coupled to cap 162 while cap 162 is secured in container holder 26. If the container is meant for one-time use, container cap 162 may be removed from container holder 26 after the procedure is complete.

Container holder 26 may be configured to be cleaned in an autoclave. In this regard, container holder 26 may be formed from materials that will not be deformed or damaged at conventional autoclave temperatures. In one embodiment, container holder 26 is made of a plastic capable of withstanding temperatures within an autoclave. Alternatively, container holder 26 may be configured to be cleaned using, for example, an alcohol bath or water and soap suitable for sanitizing medical instruments.

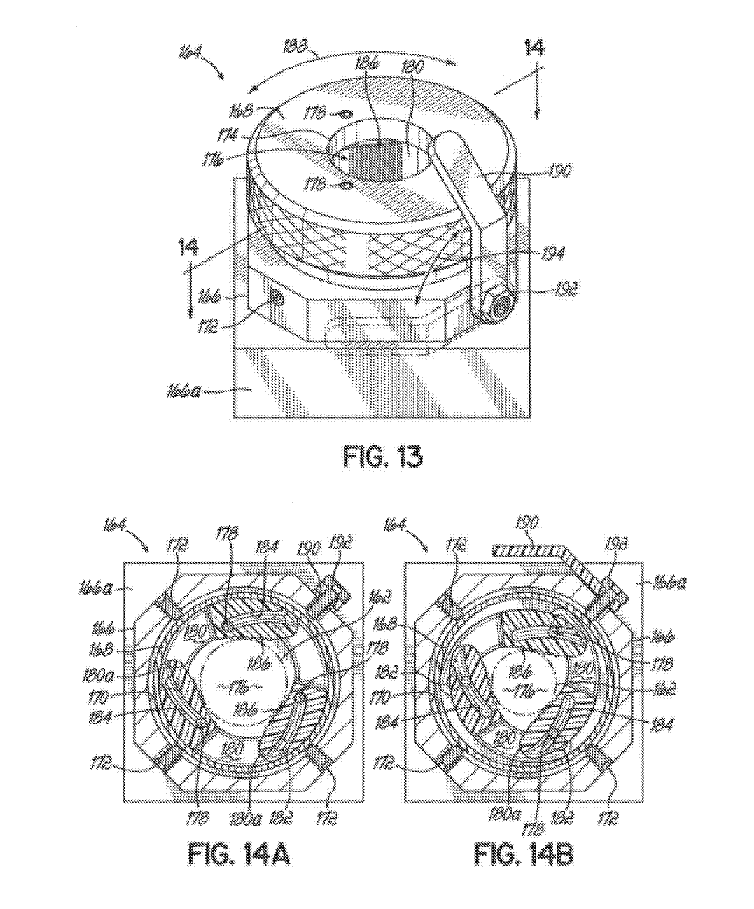

Now referring to FIGS. 13, 14A, and 14B, an embodiment of a container holder 164 is shown in detail. Container holder 164 includes a base 166 and a housing 168 rotatably coupled to base 166 via fasteners such as screws 172. Housing 168 includes an inner groove 170, and screws 172 partially extend into groove 170. By partially extending into groove 170, screws 172 prevent housing 168 being removed from base 166 and allow housing 168 to rotate. A segment 166a of base 166 has a cross-section that is larger than the size of wells 70 such that base segment 166a will not pass through the wells 70. Except for base segment 166a, container holder 164 has a cross-section that is smaller than the size of wells 70 such that, for example, housing 168 will pass through the wells 70. Housing 168 includes an aperture 174 that opens into a cavity 176. A plurality of pins 178 in cavity 176 are coupled to housing 168. Container holder 164 further includes a plurality of engagement members, or clamps 180. Clamps 180 are pivotally coupled to base 166 by respective pins 182. Each clamp 180 includes a slot 184 and includes a plurality of ridges 186. One of the pins 178 extends into each slot 184 such that each pin 178 is slidably coupled to each clamp 180. Housing 168 has a first, unengaged position where, when a container cap 162 is positioned in cavity 176, clamps 180 are not frictionally engaged with container cap 162 (FIG. 14A). Housing 168 may rotate to a second, engaged position where, when container cap 162 is positioned in cavity 176, clamps 180 are frictionally engaged with container cap 162 (arrow 188). Housing 168 has a textured surface, such as a knurled surface, to provide a better grip for the user. When housing 168 rotates to the engaged position, pins 178 slide through slots 184 and ends 180a of clamps 180 pivot about pins 182 toward a center of container holder 164. When container cap 162 is engaged in container holder 164 and a container coupled to container cap 162 by mating threads is being rotated, clamps 180 substantially prevent container cap 162 from rotating (not shown).

Container holder 164 further includes a lever 190 configured to lock the rotation of housing 168. Lever 190 is pivotally coupled to base 166 via a fastener such as screw 192. Screw 192 is aligned with groove 170 in housing 168. When lever 190 is in a first, unengaged position, screw 192 is spaced apart from groove 170 and housing 168 is allowed to rotate (FIG. 14A). Lever 190 may rotate to a second, engaged position (arrow 194). In the engaged position, screw 192 is in contact with housing 168, which prevents housing 168 from rotating (FIG. 14B). When container holder 164 is secured to instrument tray 12 by tray cover 30, the doctor may remove the container from cap 162 with one hand, which advantageously allows the doctor free to use the other hand to, for example, hold open an eyelid of the patient. If the container is multi-use, the container may be coupled to cap 162 while cap 162 is secured in container holder 164 after the doctor is finished using the container. If the container is meant for one-time use, lever 190 and housing 168 may be moved to their respective unengaged positions after the procedure is complete. Then, because clamps 180 are no longer in contact with container cap 162, container cap 162 may be removed from container holder 164.

Container holder 164 may be configured to be cleaned in an autoclave. In this regard, container holder 164 may be formed from materials that will not be deformed or damaged at conventional autoclave temperatures. In one embodiment, container holder 164 is made of aluminum and clamps 180 are made from a suitable plastic. Alternatively, container holder 164 may be configured to be cleaned using, for example, an alcohol bath or water and soap suitable for sanitizing medical instruments.

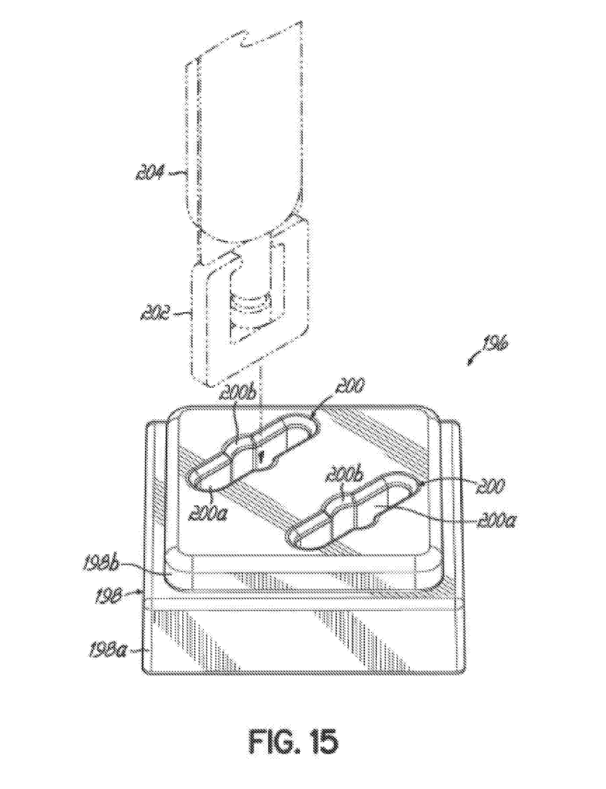

Now referring to FIG. 15, another illustrative embodiment of a container holder is shown in detail. Container holder 196 includes a base 198 and a plurality of cavities 200. A segment 198a of base 198 has a cross-section that is larger than the size of wells 70 such that base segment 198a will not pass through the wells 70. Except for base segment 198a, container holder 196 has a cross-section that is smaller than the size of wells 70 such that, for example, base segment 198b, which includes cavities 200, will pass through the wells 70. Each cavity 200 is configured to receive a cap 202. Each cavity 200 may include a wall 200a and a beveled portion 200b, which is configured to receive a portion of a container 204. When cap 202 is engaged in container holder 196 and container 204 coupled to cap 202 is being rotated, wall 200a prevents cap 202 from rotating, and beveled portion 200b allows for the rotation of container 204. Cap 202 may then be removed from container holder 196 after the procedure is complete. Container holder 196 may be configured to be cleaned in an autoclave. Alternatively, container holder 196 may be configured to be cleaned using, for example, an alcohol bath or water and soap suitable for sanitizing medical instruments.

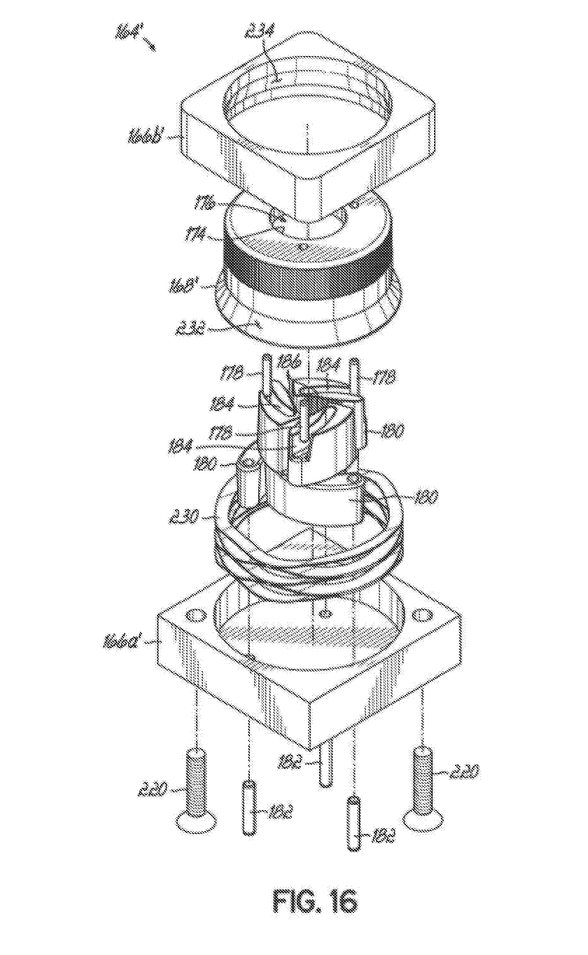

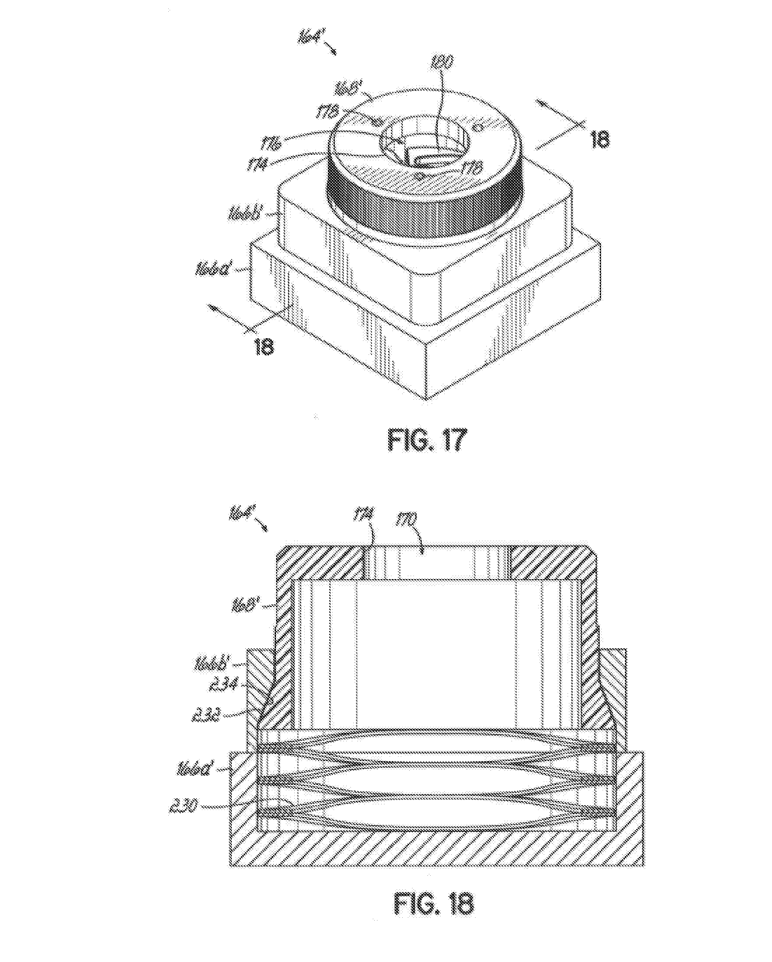

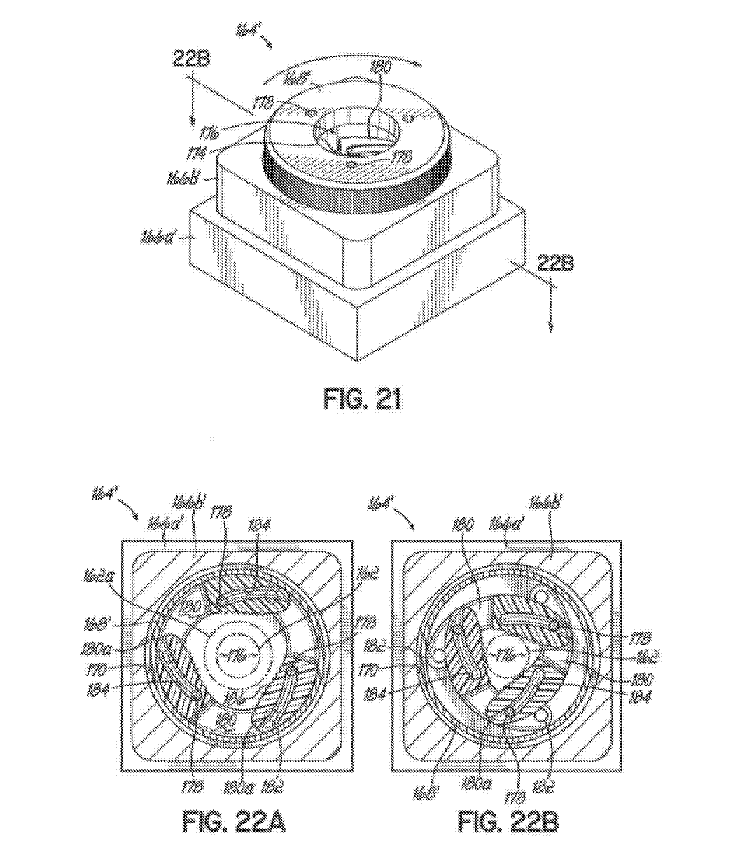

Now referring to FIGS. 16-21, 22A and 228, another embodiment of a container holder 164′ is shown in detail. It will be appreciated that container holder 164′ is very similar to container holder 164 as shown and described in connection with FIGS. 13, 14A and 148. The major difference is that the lever 190 is eliminated and a different locking mechanism is used instead, as will be described. In this embodiment, like structure as between these two embodiments will be denoted with like reference numerals and therefore may not be further described in detail. Reference numerals with prime (′) marks will be used to denote structure in the embodiment of FIGS. 16-21, 22A and 22B that has changed with regard to the previously described embodiment, and such changes shall either be described or readily apparent from the figures. Container holder 164′ includes a base 166′ and a housing 168′. The housing 168′ is rotatable relative to the base 166′. A lower segment 166a′ of base 166′ has a cross-section that is larger than the size of wells 70 such that lower base segment 166a′ will not pass through the wells 70. Lower base segment 166a′ is fixed to an upper base segment 166b by fasteners 220. Except for lower base segment 166a′, container holder 164′ has a cross-section that is smaller than the size of wells 70 such that, for example, housing 168 will pass through the wells 70. Housing 168′ includes an aperture 174 that opens into a cavity 176. A plurality of pins 178 in cavity 176 are coupled to housing 168′. Container holder 164′ further includes a plurality of engagement members, or clamps 180. Clamps 180 are pivotally coupled to base 166′ by pins 182. Each clamp 180 includes a slot 184 and includes a plurality of ridges 186. The pins 178 respectively extend into each slot 184 such that each pin 178 is slidably coupled to each clamp 180. Housing 168′ has a first, unengaged position where, when a container cap 162 is positioned in cavity 176, clamps 180 are not frictionally engaged with container cap 162. As will be appreciated by reviewing FIGS. 22A and 22B, caps 162, 162a of different sizes may be frictionally secured in between the clamps 180. FIG. 22A illustrates a first, disengaged or unlocked position of the clamps 180 that is achieved by rotating knob or housing 168′ and the attached pins 178 counterclockwise. This moves the clamps 180 radially outward to allow insertion of cap 162 or 162a. Housing 168′ is depressed and rotated clockwise to a second, engaged position as shown in FIG. 22B where, when container cap 162 is positioned in cavity 176, clamps 180 are frictionally engaged with container cap 162. Housing 168′ has a textured surface, such as a knurled surface, to provide a better grip for the user. When housing 168′ rotates to the engaged position, pins 178 slide through slots 184 and ends 180a of clamps 180 pivot about pins 182 toward a center of container holder 164′. When container cap 162 or 162a is engaged in container holder 164′ and a container coupled to container cap 162 or 162a by mating threads is being rotated, clamps 180 substantially prevent container cap 162 or 162a from rotating.

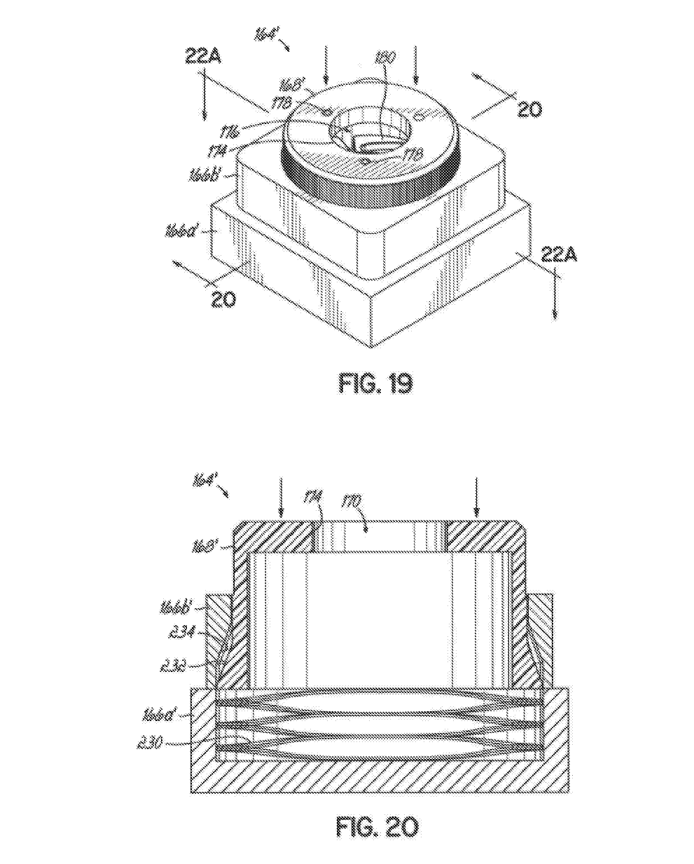

Container holder 164′ further includes a spring mechanism 230 configured to lock the rotational position of housing 168′. To rotate the housing 168′ to an unclamped position (FIG. 22A), the housing 168′ is first pushed downward as shown in FIGS. 19 and 20. This disengages surface 232 of housing 168′ from its normal frictional engagement with surface 234 of base segment 166b. The housing 168′ is allowed to return to its upper position under the bias of spring mechanism 230 to lock the engaged or clamped position (FIG. 22B). This normal frictional engagement of surfaces 232, 234 keeps housing 168′ from rotating and therefore maintains the clamping positions of clamps 180 (FIG. 22B). When the housing 168′ is pushed downward against the force of the spring mechanism 230, the housing 168′ may be rotated like a knob such that the clamps 180 are placed into their unlocked positions to release their grip on the cap 162 or 162a. On the other hand, when the cap 162 or 162a and attached bottle (not shown) are inserted into the space between the unlocked clamps 180 and the housing or knob 168′ is rotated in the opposite direction to the clamped position (FIG. 22B), the housing or knob 168′ may be released to its upper position relative to the base 166′ in which the frictional surfaces 232, 234 engage to hold the housing 168′ in position relative to the base 166′ and lock the cap 162 or 162a and attached bottle in place. When container holder 164′ is secured to instrument tray 12 by tray cover 30, the doctor may remove the container from cap 162 or 162a with one hand, which advantageously allows the doctor free to use the other hand to, for example, hold open an eyelid of the patient. If the container is multi-use, the container may be coupled to cap 162 or 162a while cap 162 or 162a is secured in container holder 164′ after the doctor is finished using the container.

Container holder 164′ may be configured to be cleaned in an autoclave. In this regard, container holder 164′ may be formed from materials that will not be deformed or damaged at conventional autoclave temperatures. In one embodiment, container holder 164′ is made of aluminum and clamps 180 are made from a suitable plastic. Alternatively, container holder 164′ may be configured to be cleaned using, for example, an alcohol bath or water and soap suitable for sanitizing medical instruments.

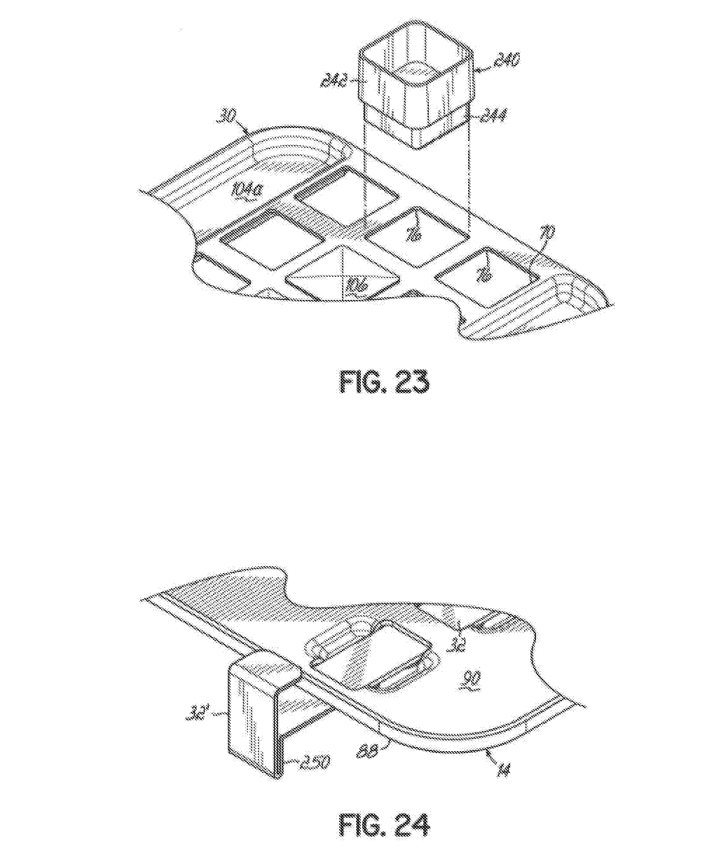

Referring now to FIG. 23, another embodiment of a well cover 240 is shown. As with the previously described well covers 106, each well cover 240 is configured to cover a well 70 when the well 70 is not engaged with an accessory. Unlike well covers 106, well cover 240 includes an upper cup portion as opposed to a flat surface. This upper cup portion 242 is easier to grasp and, if necessary or desired, may hold any suitably sized object needed by the doctor. The cup portion 242 has a cross section that is larger than the size of wells 70 such that the cup portion 242 will not pass through the wells 70. A lower portion 244 has a cross section that is smaller than the size of the wells 70 such that the lower portion 244 will pass through the wells. When a well cover 240 is positioned in a well 70, the lower portion 244 is frictionally engaged with the wall 76 of one of the wells 70, as previously described in connection with the well covers 106 shown in FIG. 6.

Referring to FIG. 24, another embodiment of a bracket 32′ is shown. This bracket 32′ is the same as bracket 32, which has been previously described, except that a downwardly extending tab 250 has been added. This tab 250 allows the bracket 32′ to more easily be grasped and slid back and forth during operation by a user.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features of the invention may be used alone or in any combination depending on the needs and preferences of the user. This has been a description of the present invention, along with the preferred methods of practicing the present invention as currently known. However, the invention itself should only be defined by the appended claims.

1. A tray cover for securing one or more accessories to an instrument tray, the tray cover comprising:
a plurality of wells,
wherein, when the tray cover is placed on the instrument tray, each of the one or more accessories is engaged by one of the plurality of wells and is secured to the instrument tray by the tray cover.

2. The tray cover of claim 1, further comprising:
a bottom surface; and
a top surface,
wherein each of the plurality of wells is open to the top and bottom surfaces and has a wall, and, when the tray cover is placed on the instrument tray, each wall of the plurality of wells is spaced apart from the instrument tray.

3. The tray cover of claim 1, wherein the tray cover has a first end and a second end, the tray cover further comprising:
two recesses, one recess of which is adjacent to the first end of the tray cover and the other recess of which is adjacent to the second end of the tray cover.

4. An instrument tray assembly for use during a medical procedure, the assembly comprising:
an instrument tray; and
a tray cover having a plurality of wells, the tray cover configured to be placed on the instrument tray,
wherein, when one or more accessories are positioned on the instrument tray and the tray cover is placed on the instrument tray, each of the one or more accessories is engaged by one of the plurality of wells and is secured to the instrument tray by the tray cover.

5. The assembly of claim 4, wherein the tray cover has a bottom surface and a top surface, and wherein each of the plurality of wells opens to the bottom and top surfaces, and, when the one or more accessories are positioned on the instrument tray and the tray cover is positioned on the instrument tray, each of the one or more accessories extends through one of the plurality of wells.

6. An instrument tray assembly for use during a medical procedure, the assembly comprising:
a base tray;
an instrument tray, the instrument tray configured to be positioned on the base tray;
a tray cover having a plurality of wells, the tray cover configured to be positioned on the instrument tray; and
a locking mechanism configured to secure the tray cover to the base tray,
wherein, when one or more accessories are positioned on the instrument tray and the tray cover is positioned on the instrument tray, each of the one or more accessories is engaged by one of the plurality of wells and is secured to the instrument tray by the tray cover.

7. The assembly of claim 6, wherein the tray cover has a bottom surface and a top surface, and wherein each of the plurality of wells opens to the bottom and top surfaces, and, when the one or more accessories are positioned on the instrument tray and the tray cover is secured to the base tray, each of the one or more accessories extends through one of the plurality of wells and is secured to the instrument tray.

8. The assembly of claim 6, wherein the base tray and the instrument tray each have a peripheral edge, and wherein the peripheral edge of the base tray is configured to be flush with the peripheral edge of the instrument tray when the instrument tray is positioned on the base tray.

9. The assembly of claim 6, wherein the locking mechanism includes one or more brackets configured to prevent relative movement between the base tray, the instrument tray, and the tray cover.

10. An instrument stand assembly for use during a medical procedure, the assembly comprising:
a base;
a support arm extending from the base;
a base tray removably mounted to the support arm;
an instrument tray configured to be placed on the base tray;
a tray cover having a plurality of wells, the tray cover being configured to be placed on the instrument tray; and
a locking mechanism configured to secure the tray cover and the instrument tray to the base tray,
wherein, when one or more accessories and the tray cover are positioned on the instrument tray and the tray cover is secured to the base tray, each of the one or more accessories is engaged by one of the plurality of wells and is secured to the instrument tray by the tray cover.

11. The assembly of claim 10, wherein the locking mechanism includes one or more brackets configured to prevent relative movement between the base tray, the instrument tray, and the tray cover.

12. The assembly of claim 10, wherein the tray cover has a bottom surface and a top surface, and wherein each of the plurality of wells opens to the bottom and top surfaces, and, when one or more accessories and the tray cover are positioned on the instrument tray and the tray cover is secured to the base tray, each of the one or more accessories extends through one of the plurality of wells.

13. The assembly of claim 10, wherein the base tray and the instrument tray each have a peripheral edge, and wherein the peripheral edge of the base tray is configured to be flush with the peripheral edge of the instrument tray when the instrument tray is positioned on the base tray.

14. The assembly of claim 10, further comprising:
a support pole extending from the base,
wherein the support arm is movably secured to the support pole.

15. The assembly of claim 10, wherein the support arm has a first end and a second end, the first end being coupled to the base and the support arm being configured to allow the second end to articulate relative to the first end.

16. The assembly of claim 15, wherein the support arm is configured to support one or more accessories and retain an orientation of the one or more accessories relative to the base when the second end articulates.

17. The assembly of claim 10, wherein the support arm is configured to support one or more accessories.

18. The assembly of claim 17, wherein the assembly further comprises:
an accessory holder coupled to the support arm configured to secure an accessory to the support arm.

19. The assembly of claim 18, wherein the accessory holder is a sharps disposal container holder.

20. The assembly of claim 18, wherein the accessory holder is a tissue box holder.
